(19) United States
(12) Patent Application Publication
(10) Pub. No.: US 2026/0053428 A1
(43) Pub. Date: Feb. 26, 2026

(54) SYSTEMS AND METHODS FOR HAIR AND SCALP HEALTH ANALYSIS

(71) Applicant: FITSKIN INC., Toronto (CA)

(72) Inventors: Sergio RATTNER, Toronto (CA); Justinas VILIMAS, Vilnius (LT)

(21) Appl. No.: 19/112,896

(22) PCT Filed: Sep. 20, 2023
(86) PCT No.: PCT/CA2023/051247

§ 371 Date: Mar. 18, 2025

Related U.S. Application Data
(60) Provisional application No. 63/408,458, filed on Sep. 20, 2022.

(51) Int. Cl. A61B 5/00; G06T 7/00

(52) U.S. Cl. CPC A61B 5/446; A61B 5/0064; A61B 5/0077; A61B 5/0082; G06T 7/0012; G06T 2207/10004; G06T 2207/20081; G06T 2207/30088

(57) ABSTRACT

There is a system for one or more of a user hair health and scalp health determination of a user, the system comprising: a hair analysis assembly, configured to: obtain a set of user head images of the user; analyze the set of user head images to detect one or more head characteristics; calculate a set of user head health scores, based on the detected one or more head characteristics. The system may further output the user head health score.

### TECHNICAL FIELD

The present invention relates to measurement and analysis of hair and scalp characteristics and health using hair analysis devices that attach to smartphones.

### BACKGROUND

Hair and scalp care product manufacturers create products to assist users with maintaining healthy hair and scalps. However, one of the biggest problems is determining accurate and useful scores for the hair and scalp, to be able to properly match and recommend products and treatments.

Some solutions do exist that attempt to determine an accurate and useful analysis. However, limitations and failures of these solutions abound. For example existing solutions suffer from inaccuracies of measurement, variability based on the user or situation, high cost, expertise required to operate the solution or perform the analysis, and logistics challenges such as bulky hardware.

There is accordingly a need in the art for an improved method and system capable of determining accurate hair health and scalp health (sometimes collectively referred to herein as “head health”) assessments or scores.

### SUMMARY OF THE INVENTION

There is a system for head health assessment of a user, the system comprising: a hair analysis assembly, configured to: receive an input to initiate the capture of a first set of head images of a user; obtain the first set of head images; send the first set of head images to a trained artificial intelligence system for analysis of the first set of head images for a first set of head health features; receive, from the trained artificial intelligence system, a first set of head health feature results comprising the locations and quantities of each of the first set of head health features in each image in the first set of user head images; apply a first set of head health rules to the first set of head health feature results; arrive at a first set of head health assessments based on the applying; and report the first set of head health assessments.

The hair analysis assembly may comprise a mobile device and a hair analysis device that removably attaches to the mobile device.

The hair analysis assembly may further be configured to receive a product recommendation based on the first set of head health assessments and report the product recommendation.

The first set of head images may comprise a first set of hair images and the first set of head health features may comprise a first set of hair health features.

The first set of hair health features may comprise thermal damage, mechanical damage and dryness.

The head health rules may comprise hair health rules wherein the hair health rules comprise comparing the quantities of each of the first set of hair health features in the hair images to a configurable threshold for each of the first set of hair health features.

The first set of head images may comprise a first set of scalp images and the first set of head health features may comprise a first set of scalp health features.

The first set of scalp health features may comprises small flakes, medium and large flakes, flaky hair shaft, flaky scalp oil pooling, gooey hair shaft, and build-up.

The head health rules may comprise scalp health rules wherein the scalp health rules may comprise comparing the quantities of each of the first set of scalp health features in the scalp images to a configurable threshold for each of the first set of scalp health features.

The hair analysis device may further comprise a cross polarized light, and wherein the obtaining may be with the cross polarized light on for a first subset of the first set of scalp health features.

The first set of head images may comprise a first set of hair images and a first set of scalp images and the first set of head health features comprises a first set of hair health features and a first set of scalp health features.

The first set of hair health features may comprise thermal damage, mechanical damage and dryness and the first set of scalp health features comprises small flakes, medium and large flakes, flaky hair shaft, flaky scalp oil pooling, gooey hair shaft, and build-up.

The head health rules comprise hair health rules, wherein the hair health rules may comprise comparing the quantities of each of the first set of hair health features in the hair images to a configurable threshold for each of the first set of hair health features, and scalp health rules wherein the scalp health rules may comprise comparing the quantities of each of the first set of scalp health features in the scalp images to a configurable threshold for each of the first set of scalp health features.

The obtaining further may comprise preparing the head analysis device for the head images to be obtained and head characteristics to be assessed by establishing a capabilities setup therefor.

There is also a method for head health assessment of a user, the method comprising: receiving, by a hair analysis assembly, an input to initiate the capture of a first set of head images of a user; obtaining, by the hair analysis assembly, the first set of head images; sending the first set of head images to a trained artificial intelligence system for analysis of the first set of head images for a first set of head health features; receiving, by the hair analysis assembly from the trained artificial intelligence system, a first set of head health feature results comprising the locations and quantities of each of the first set of head health features in each image in the first set of user head images; applying a first set of head health rules to the first set of head health feature results; arriving at a first set of head health assessments based on the applying; and report the first set of head health assessments.

The first set of head images may comprise a first set of hair images and the first set of head health features comprises a first set of hair health features.

The first set of hair health features may comprise thermal damage, mechanical damage and dryness.

The head health rules may comprise hair health rules wherein the hair health rules may comprise comparing the quantities of each of the first set of hair health features in the hair images to a configurable threshold for each of the first set of hair health features.

The first set of head images may comprise a first set of scalp images and the first set of head health features may comprise a first set of scalp health features.

The first set of scalp health features may comprise small flakes, medium and large flakes, flaky hair shaft, flaky scalp oil pooling, gooey hair shaft, and build-up.

The head health rules may comprise scalp health rules wherein the scalp health rules may comprise comparing the quantities of each of the first set of scalp health features in the scalp images to a configurable threshold for each of the first set of scalp health features.

The hair analysis device may further comprise a cross polarized light, and wherein the obtaining may be with the cross polarized light on for a first subset of the first set of scalp health features.

The first set of head images may comprises a first set of hair images and a first set of scalp images and the first set of head health features comprises a first set of hair health features and a first set of scalp health features.

The first set of hair health features may comprise thermal damage, mechanical damage and dryness and the first set of scalp health features comprises small flakes, medium and large flakes, flaky hair shaft, flaky scalp oil pooling, gooey hair shaft, and build-up.

The head health rules may comprise hair health rules, wherein the hair health rules may comprise comparing the quantities of each of the first set of hair health features in the hair images to a configurable threshold for each of the first set of hair health features, and scalp health rules wherein the scalp health rules may comprise comparing the quantities of each of the first set of scalp health features in the scalp images to a configurable threshold for each of the first set of scalp health features.

The obtaining may further comprise preparing the head analysis device for the head images to be obtained and head characteristics to be assessed by establishing a capabilities setup therefor.

There is a system for one or more of a user hair health and scalp health determination of a user, the system comprising: a hair analysis assembly, configured to: obtain a set of user head images of the user; analyze the set of user head images to detect one or more head characteristics; calculate a set of user head health scores, based on the detected one or more head characteristics. The system may further output the user head health score.

The hair analysis assembly may comprise a mobile device and a hair analysis device that attaches to a mobile device.

The hair analysis device may further comprise a cross polarized light, wherein the cross polarized light is used to obtain at least one of the user head images and the analyzing includes the at least one of the user head images obtained using the cross polarized light.

The head characteristics may be one or more of a set of hair characteristics, which may comprise thermal damage, mechanical damage, dryness and a set of scalp characteristics, which may comprise small flakes, medium and large flakes, flaky hair shaft, flaky scalp oil pooling, gooey hair shaft, and build-up.

The calculating may be further based on one or more hair analysis rules and scalp analysis rules.

The set of user head health scores may comprise one or more hair scores and one or more scalp scores.

The hair analysis assembly may be further configured to provide a product recommendation, based on the user head health scores.

There is also a method for one or more of a user hair health and scalp health determination of a user, the method comprising: obtaining, from a hair analysis assembly, a set of user head images of the user; analyzing the set of user head images to detect one or more head characteristics; calculating a set of user head health scores, based on the detected one or more head characteristics; and outputting the user head health score.

The hair analysis assembly may comprise a mobile device and a hair analysis device that attaches to a mobile device.

The hair analysis device may further comprise a cross polarized light, wherein the cross polarized light is used to obtain at least one of the user head images and the analyzing includes the at least one of the user head images obtained using the cross polarized light.

The head characteristics may be one or more of a set of hair characteristics, which may comprise thermal damage, mechanical damage, dryness and a set of scalp characteristics, which may comprise small flakes, medium and large flakes, flaky hair shaft, flaky scalp oil pooling, gooey hair shaft, and build-up.

The calculating may be further based on one or more hair analysis rules and scalp analysis rules.

The set of user head health scores may comprise one or more hair scores and one or more scalp scores.

The method may further comprise providing a product recommendation, based on the user head health scores.

### BRIEF DESCRIPTION OF THE DRAWINGS

The invention is illustrated in the figures of the accompanying drawings which are meant to be exemplary and not limiting, in which like references are intended to refer to like or corresponding parts, and in which:

FIG. 1 illustrates aspects of a hair analysis system, according to an embodiment of the present invention;

FIG. 2 is a method for hair health determination using a hair analysis system, according to embodiments of the present invention; and

FIGS. 3a-d are exemplary screenshots from an application embodying aspects of the present invention;

FIGS. 4a-c are hair images that show exemplary features to assist with hair health assessments;

FIGS. 5a-g are scalp images that show exemplary features to assist with scalp health assessments;

FIG. 6 is a hair image that shows identification of exemplary features relating to hair health assessments; and

FIG. 7 is a hair image that shows identification of exemplary features relating to hair health assessments.

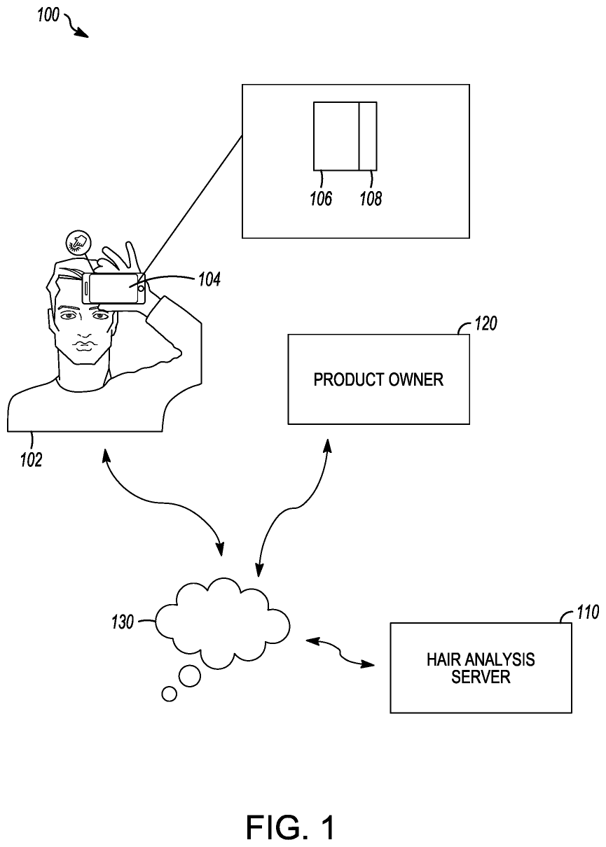

### DETAILED DESCRIPTION OF THE INVENTION

Broadly, system 100 comprises a hair analysis assembly 104 (“HAA”, which may comprise a hair analysis device 108—“scanner”—that attaches, preferably removably attaches, to a mobile device 106) that, when used by a user 102, performs one or more hair analysis actions such as capturing images of a user's hair or face for color assessment, as described herein.

HAA 104 may be as described in PCT/CA2020/050216 or PCT/CA2017/050503, or may comprise another hair analysis system that is capable of taking images of a user's head, the images having characteristics that are sufficient for the analysis described herein. It is to be understood that the hair analysis assembly 104 may have similar hardware components as described therein, and be able to interact and function similarly to as described in such references. HAA may have an application (app) thereon that user 102 can use to enable, control, or review methods described herein.

Notably, and as mentioned, system 100 requires the ability to obtain head images (hair images and/or scalp images) that allow the processing described herein. In one embodiment, head images may be taken using cross polarized light or while the cross polarized light is “on” (for example to remove glare, or reflection of the light source from the head image), with a 10 megapixel camera at a magnification of not less than 10×.

User head images may be in one of several color formats, such as LAB or RGB. User head images can be substantially of any quality, type, format or size/file size, provided the methods herein can be applied. For example, images may be compressed or not compressed, raw or processed, and in a variety of file formats.

Hair analysis server (HAS) 110 may be a server that stores and processes head characteristic measurement or sample, as described herein. HAS 110 may be any combination of web servers, applications servers, and database servers, as would be known to those of skill in the art. Each of such servers may comprise typical server components including processors, volatile and non-volatile memory storage devices and software instructions executable thereon. HAS 110 may communicate via app 18 to perform the functionality described herein, including exchanging head images, product recommendations, e-commerce capabilities, and the like. Of course, app may perform these, alone or in combination with HAS 110, as well.

HAS 110 may include a database server that receives and stores all head characteristic samples from all users into a user profile for each registered user 102 and guest user 102. These may be received from one or more HAA 104, though app may be configurable to store head images locally only (though that may preclude some of the results information based on population and demographic comparisons).

HAS 110 may provide various analysis functionality as described herein (such as computing histograms of comparisons with a user's historical results or of comparisons with peers) and may provide various display functionality as described herein (such as providing websites that may present various analysis, provide links or functional links for other websites to access and display such results, recommendations and the like).

Product owners 120 may be entities that have hair and scalp care products, for example that can, or should, be adapted or selected based on a user's head. Product owners 120 may also have one or more product owner servers including web servers, applications servers, and database servers, as would be known to those of skill in the art. Each of such servers may comprise typical server components including processors, volatile and non-volatile memory storage devices and software executable thereon. Product owner 120 may be a point of communication for app 18 (directly, or via HAS 110) for hair analysis measurement samples (such as those obtained via a user that was provided hair analysis device 20 by such product owner 120) and for storage and execution of product recommendation algorithms. For example, one or more generic product recommendation algorithms may be stored and owned by HAS 110 for each product recommendation type, and product owners may own and implement their own proprietary product recommendation algorithms (for example with product owner 120 receiving the required data to perform the product recommendation algorithm and returning the recommended product).

Product owners 120 may also offer e-commerce services directly, may suggest vendors (not shown) such as Amazon™ (separately or with the recommended products) or may be agnostic about how a user may purchase a recommended product. Product owners 120 may also provide one or more e-commerce websites or screens (separate from or embedded in app) that facilitate business or commercial transactions involving the transfer of information over network 130 (such as the Internet). Types of e-commerce sites include but are not limited to: retail sites, auctions sites, and business-to-business sites. Exemplary vendors that may facilitate the purchase of head care products may include Amazon™ eBay™, and Overstock™. Of course product owners 120 may have their own e-commerce sites as part of their general websites, or HAS 110 may be such a vendor.

There may be other users 102 involved in system 102, such as beauty advisors or consultants, for example that work for product owners 120 or commerce sites or stores, that may assist a user 102 who is the subject of the image and whose head health is being determined.

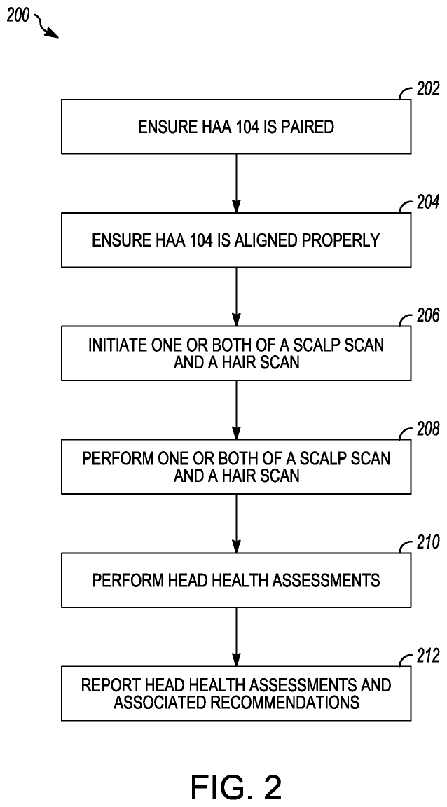

Turning to FIG. 2 there is a method 200 for head (hair and/or scalp) health determination and processing.

Method 200 begins at 202 when pairing occurs.

Hair analysis device 108 may have an SDK that an app on mobile device 104 can use to access functionality of HAD 108. The SDK may provide methods to discover, pair and control the scanner.

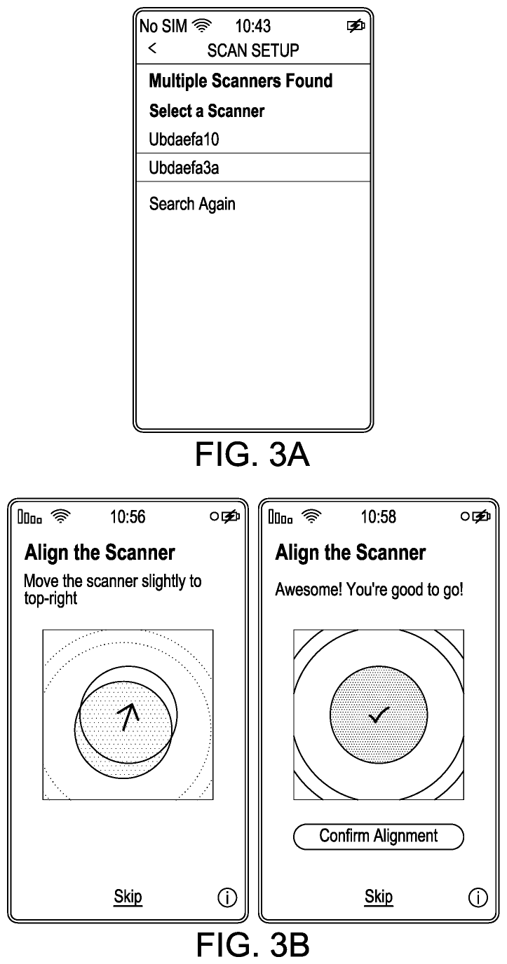

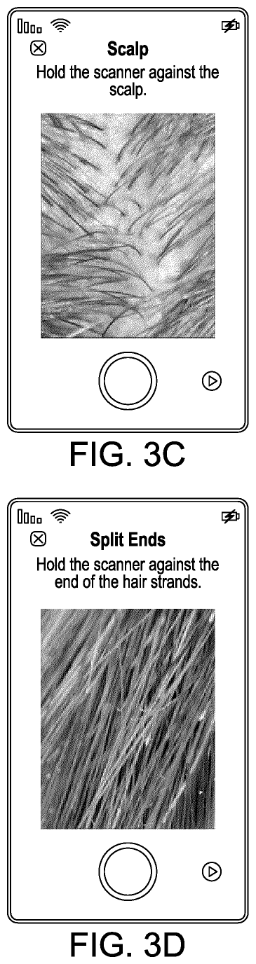

On the first app launch it is recommended to scan, discover and display all the scanners in range (using the scanForDevices method). If after a scanning session only a single scanner is discovered, consider initiating the pairing automatically (connectToDevice method) without displaying a list of discovered scanners. See FIG. 3a.

After pairing is successful, for a better user experience, the ID of the paired device may be stored. Then on every consecutive app launch, reconnecting to that device may be done automatically, without repeating the pairing procedure.

Upon app resigning or leaving an active state (smartphone going to sleep or the app moved to background), the SDK can automatically disconnect from the scanner. When the app is brought back to foreground, the SDK can reconnect to the paired scanner. Note the app should always check if a valid connection is available when performing scanner related actions. Such validation may be part of pairing at 202.

Method 200 continues at 204 where alignment occurs. This may be omitted if the scanner is removably attached to mobile device in a way that guarantees proper alignment.

For clip-on scanner models the SDK may provide a helper method to guide the user with the alignment of the scanner. A scanner needs to be clipped on the correct camera lens and centered accurately to negate any optical distortion or obscuration. A method startLensPositioningValidation can be used to start tracking the location of the scanner. This method provides constant status updates regarding alignment information. After the alignment is confirmed by the user, endLensPositioningValidation method needs to be called to stop the alignment status updates. It may help to turn on an LED white light when displaying a camera live view. See FIG. 3b.

At 206 and 208 one or more head health assessments or head health scans are initiated and performed, where one or more sets of head images are obtained.

Scanner SDK provides a method takeScalpPhoto method to initiate the capturing of a user's scalp. Usually this method should be triggered via a button click, or some other form of user input, to make sure a user is able to position a scanner on a scalp (their own or their customer's). It is recommended to display a live view of the camera as well. See FIG. 3c for an exemplary live view set up for a scalp image capture.

Scanner SDK provides a method takeHairPhoto method to initiate the capturing of a user's hair. Usually, this method should be triggered via a button click, to make sure a user is able to position a scanner against the end of the hair strands (their own or their customer's). It is recommended to display a live view of the camera as well. See FIG. 3d for an exemplary live view set up for a hair image capture.

Initiating a scalp image capture and a hair image capture do not typically occur at the same time given the different distances focus for each of the head image captures (hair image capture and scalp image capture). As such, as part of 206 and/or 208, HAA may prepare for the particular head image capture that is being initiated (for example by setting the right focus distance, turning on the right light, adjusting to the right magnification, and the like—ie establishing a capabilities setup that applies for the head image type—hair or scalp—and head characteristic—hair characteristic(s) or scalp characteristics(s)). However, capturing a set of hair images and a set of scalp images can occur in any order.

Additionally, each head image capture may include capturing one or more images, using one or more capabilities of the scanner. For example, different lighting, flash, and elements/settings of a camera (magnification, focus distance, lights used, for example) might be used. Each collection of settings for the capabilities may be referred to as a capability setup and may relate to a particular type of head image to be captured, and head characteristic to be assessed. As may be further discussed, cross polarized light may be used in one scalp image capture (for example as it helps identify some of the features that are detected to assess scalp health, namely flakes on the scalp and flaky hair shafts. It is to be understood that various combinations of images, taken with various combinations of settings, are within the scope of the present invention, depending on the desired health features to detect, useability, hardware of the scanner, and the like.

Image analysis occurs at 210 and comprises hair health analysis (analyzing one or more images of the user's hair, as described herein) and scalp health analysis (analyzing one or more images of the user's scalp, as described herein).

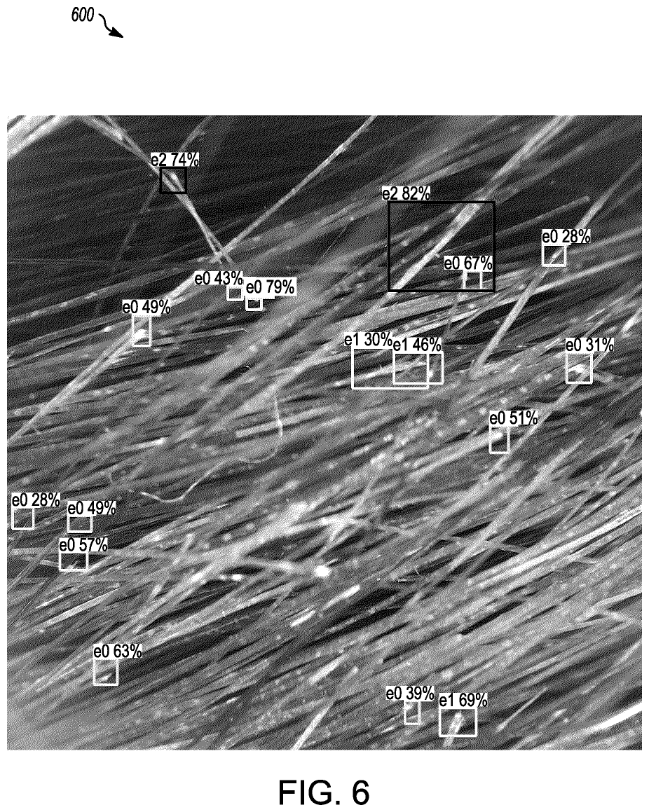

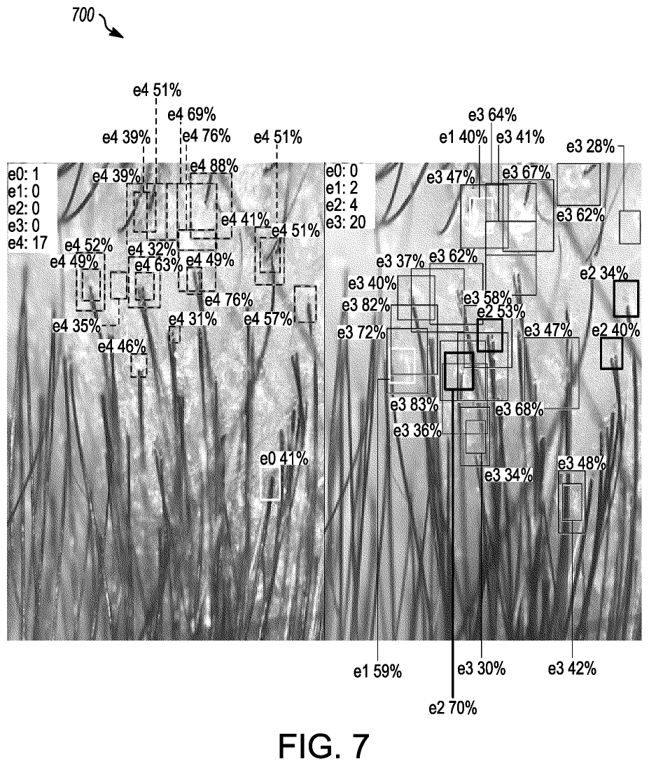

Analysis may be performed by providing the appropriate user head images to an artificial intelligence system (such as a common object detection ML model, for example YOLOv2) that has been trained to identify one or more hair health features and/or scalp health features (hair health features and scalp health features being examples of head health features). Such AI system may review the images and locate and quantify the occurrences of such hair health features such as those shown in FIGS. 6 and 7 (as described herein, in head image 600 (hair image) and 700 (scalp image)). After identifying the head health features, various head health rules (hair health rules and scalp health rules) may be applied to arrive at head health assessments, such as a hair health assessment/score and a scalp health assessment/score—as described herein. The trained artificial intelligence system may be local (for example on a mobile device) or remote from the hair analysis assembly 104.

Exemplary hair health features and rules and/or scalp health features and rules, are described below, but others are possible.

### Hair Health Analysis (Split Ends) and Hair Health Features/Characteristics

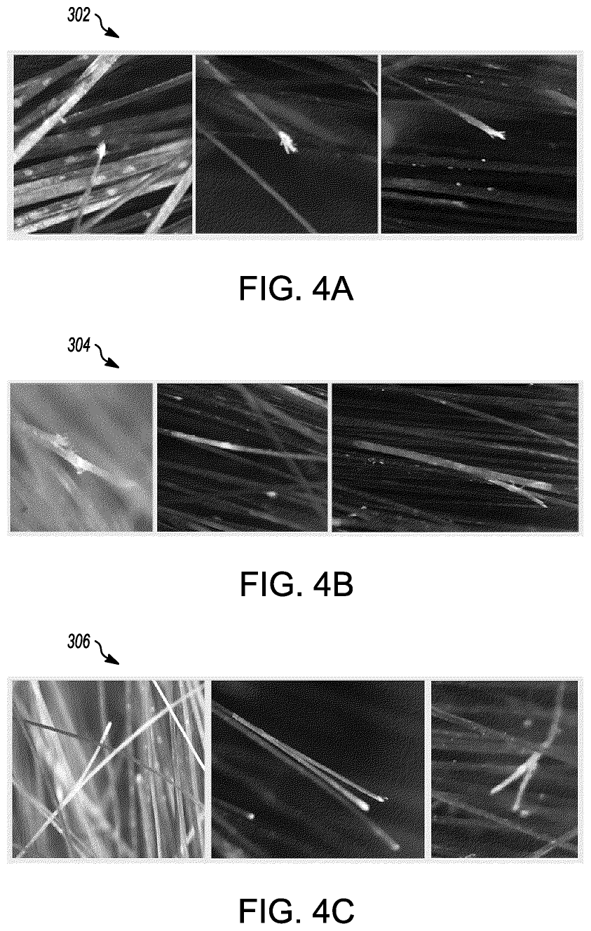

Processing and output is detection for (count or number, location and size or severity) the features/characteristics of thermal damage (see FIG. 4a—may be caused by hair being exposed to high heat, such as from blow dryers, flat irons, etc., and results in weaker hair that loses its elasticity and is more prone to damage), mechanical damage (see FIG. 4b—may be caused by detangling techniques used incorrectly, tension and over-manipulation and results in thinning hair edges, knots and excessive splitting and breakage) and dryness (see FIG. 4c—may be caused by dry climate and washing your hair too often with too-hot water or lack of heat protection due to blow dryers, flat irons, etc., where dry hair may result in brittle hair, which causes splitting hair ends). The algorithms may output a damage assessment (user hair score) of thermal, mechanical, dry (or some combination thereof) or none, according to one or more configurable assessment rules.

Exemplary hair analysis or assessment rules include:
(a) If “thermal damage” feature detection count is over 4 (or some other configurable number/threshold), thermal damage is selected as a/the final analysis assessment; ELSE
(b) If “mechanical damage” feature detection count is over 4 (or some other configurable number/threshold), mechanical damage is selected as a/the final analysis assessment; ELSE
(c) If “dryness” feature detection count is over 4 (or some other configurable number/threshold), dry damage is selected as a/the final analysis assessment; ELSE
(d) None.

It is to be understood that each number above (for example a count above “4”) may be configurable and may change based on parameters that establish what constitutes the particular feature detection. In addition, other assessment rules may be chosen, for example by running the system to identify damage, and basing rules on observed (human or machine driven) results.

It is further to be understood that various features (hair and/or scalp), and combinations of features (hair and/or scalp), may be assessed for any given set of head images, and some or all of the selected features may be used for the various rules that may be used to make one or more assessments. In one embodiment thermal damage, mechanical damage and dryness are all features that hair images are analyzed for, and all factor into the rules to assess hair health. But various permutations and combinations are possible.

### Scalp Health Analysis and Scalp Health Features/Characteristics

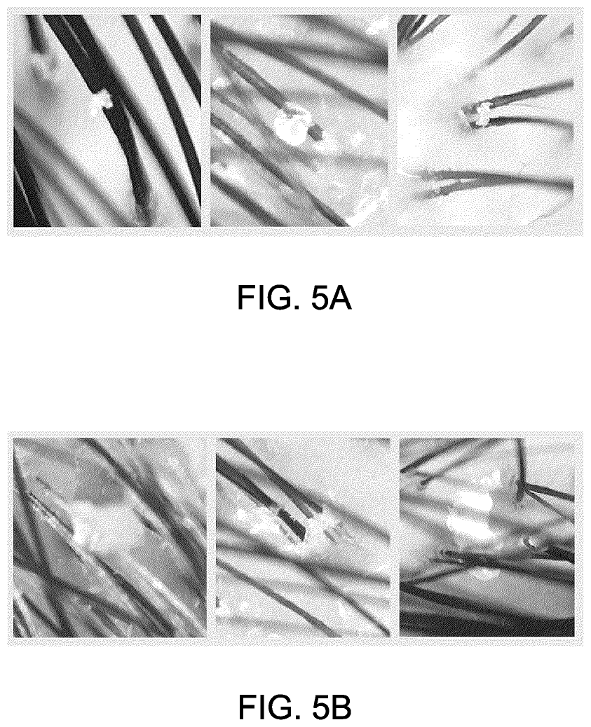

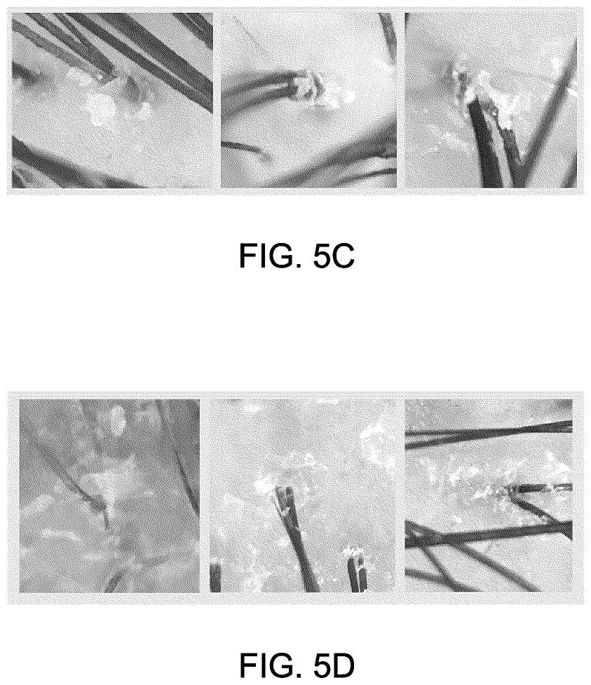

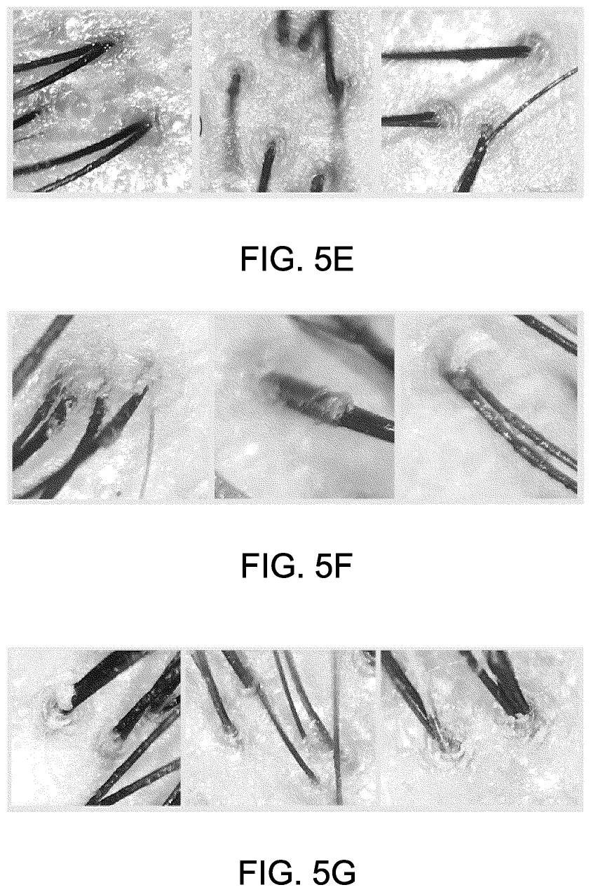

Processing and output is detection for (count or number, location and size or severity) the features/characteristics of small flakes (see FIG. 5a—preferably using cross polarized image, where a small flake is a small piece of dead scalp skin, generally no larger than 1 mm in diameter, indicating scalp dryness), medium & large flakes (see FIG. 5b—using cross polarized image, where medium and large flakes are tiny pieces of dead scalp skin, larger than 1 mm in diameter and are treated separately from small flakes because small flakes tend to indicate scalp dryness, while medium & large flakes tend to indicate dandruff), flaky hair shaft (see FIG. 5c—preferably using cross polarized image, where flaky hair shaft is an unhealthy hair shaft riddled with tiny pieces of dead scalp skin, which indicates scalp dryness, which may be separately identifiable by AI tools and thus used separately from other flake types), flaky scalp (see FIG. 5d—preferably using cross polarized image, where flaky scalp is a tiny piece of dead scalp skin that is still partly attached to the scalp and is in progress of becoming a large flake. As with flaky hair shaft, this formation has unique visual features, mainly being semi-transparent and never attached to a hair shaft, which may require it being treated as a separate feature by the ML model.), oil pooling (see FIG. 5e—preferably not using cross polarized image, where oil pooling is a scalp condition when excessive oil is accumulating around a hair shaft. It can be described as semi-transparent circles forming around a hair shaft. Presence of these features indicate scalp oiliness), gooey hair shaft (see FIG. 5f—preferably not using cross polarized image, where gooey hair shaft is a scalp oiliness feature indicating extreme excess of oil around a hair shaft which can be described as semi-transparent mass of material accumulated around a hair shaft and presence of these features may indicate extreme scalp oiliness), and build-up (see FIG. 5g—not using cross polarized image, where build-up can be caused by both excessive oiliness and scalp dryness and on rare cases it can be caused by product build-up). While build-up is visually similar to “gooey hair shaft”, a distinction can be made because it's not typically semi-transparent and generally has tiny particles around the hair shaft). The algorithms may output a damage assessment of oily, build-up, flaky or normal, according to one or more configurable assessment rules.

Exemplary scalp assessment or analysis rules include:
(a) If “oil pooling”+“Gooey hair shaft” feature detection count is equal or over 4, oily damage is selected as the final analysis assessment; ELSE
(b) If “build-up” feature detection count is equal or over 2 AND “Sum of all flake detection” (“Small flakes”+“Medium & large flakes”+“Flaky hair shaft”+“Flaky scalp”) is below “Build-up” count multiplied by 5, build-up is selected; ELSE
(c) If “sum of all flake detection” (“Small flakes”+“Medium & large flakes”+“Flaky hair shaft”+“Flaky scalp”) feature detection count is over 4 OR “Flaky scalp” is equal or over 1, flaky is selected; ELSE
(d) None.

It is to be understood that each number above (for example a count above “4”) may be configurable and may change based on parameters that establish what constitutes the particular feature detection. In addition, other assessment rules may be chosen, for example by running the system to identify damage, and basing rules on observed (human or machine driven) results.

It is further to be understood that various features (hair and/or scalp), and combinations of features (hair and/or scalp), may be assessed for any given set of head images, and some or all of the selected features may be used for the various rules that may be used to make one or more assessments. In one embodiment oil pooling, gooey hair shaft, small flakes, medium & large flakes, flaky hair shaft, and flaky scalp, are all features that scalp images are analyzed for, and all factor into the rules to assess scalp health. But various permutations and combinations are possible.

At 212, head health assessments and head health assessment data (which may include the underlying user head images, processed user head images showing the detected features, scoring and assessments) can be reported or saved (for example, displayed on mobile device, sent to cloud storage such as product owner 120 or hair analysis server 110, and the like). In addition to reporting and saving, users may be provided one or more product recommendations that may help with their head health, to deal with a head health assessment. Recommendations may be made by a product owner as well, based on their knowledge of how their products impact head health features and by a user answering questions about their experiences and goals.

The above-described embodiments of the present disclosure can be implemented in any of numerous ways. For example, the embodiments may be implemented using hardware, software or a combination thereof. When implemented in software, the software code can be executed on any suitable processor or collection of processors, whether provided in a single computer or distributed among multiple computers.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the concepts disclosed herein may be embodied as a non-transitory computer-readable medium (or multiple computer-readable media) (e.g., a computer memory, one or more floppy discs, compact discs, optical discs, magnetic tapes, flash memories, circuit configurations in Field Programmable Gate Arrays or other semiconductor devices, or other non-transitory, tangible computer storage medium) encoded with one or more programs that, when executed on one or more computers or other processors, perform methods that implement the various embodiments of the present disclosure discussed above. The computer-readable medium or media can be transportable, such that the program or programs stored thereon can be loaded onto one or more different computers or other processors to implement various aspects of the present disclosure as discussed above.

The terms “program”, “app” or “application” or “software” are used herein to refer to any type of computer code or set of computer-executable instructions that can be employed to program a computer or other processor to implement various aspects of the present disclosure as discussed above. Additionally, it should be appreciated that according to one aspect of this embodiment, one or more computer programs that when executed perform methods of the present disclosure need not reside on a single computer or processor, but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

Various features and aspects of the present disclosure may be used alone, in any combination of two or more, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the concepts disclosed herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc. in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

Several (or different) elements discussed below, and/or claimed, are described as being “coupled”, “in communication with”, or “configured to be in communication with”. This terminology is intended to be non-limiting, and where appropriate, be interpreted to include without limitation, wired and wireless communication using any one or a plurality of a suitable protocols, as well as communication methods that are constantly maintained, are made on a periodic basis, and/or made or initiated on an as needed basis.

Embodiments may also be implemented in cloud computing environments. In this description and the following claims, “cloud computing” may be defined as a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

This written description uses examples to disclose the invention and also to enable any person skilled in the art to make and use the invention. The patentable scope of the invention is defined by the claims, and may include other examples that occur to those skilled in the art. Such other examples are within the scope of the claims if they have structural elements that do not differ from the literal language of the claims, or if they include equivalent structural elements with insubstantial differences from the literal language of the claims.

It may be appreciated that the assemblies and modules described above may be connected with each other as required to perform desired functions and tasks within the scope of persons of skill in the art to make such combinations and permutations without having to describe each and every one in explicit terms. There is no particular assembly or component that may be superior to any of the equivalents available to the person skilled in the art. There is no particular mode of practicing the disclosed subject matter that is superior to others, so long as the functions may be performed. It is believed that all the crucial aspects of the disclosed subject matter have been provided in this document. It is understood that the scope of the present invention is limited to the scope provided by the independent claim(s), and it is also understood that the scope of the present invention is not limited to: (i) the dependent claims, (ii) the detailed description of the non-limiting embodiments, (iii) the summary, (iv) the abstract, and/or (v) the description provided outside of this document (that is, outside of the instant application as filed, as prosecuted, and/or as granted). It is understood, for this document, that the phrase “includes” is equivalent to the word “comprising.” The foregoing has outlined the non-limiting embodiments (examples). The description is made for particular non-limiting embodiments (examples). It is understood that the non-limiting embodiments are merely illustrative as examples.

What is claimed is:

1. A system for head health assessment of a user, the system comprising:
a hair analysis assembly, configured to:
receive an input to initiate the capture of a first set of head images of a user;
obtain the first set of head images;
send the first set of head images to a trained artificial intelligence system for analysis of the first set of head images for a first set of head health features;
receive, from the trained artificial intelligence system, a first set of head health feature results comprising the locations and quantities of each of the first set of head health features in each image in the first set of user head images;
apply a first set of head health rules to the first set of head health feature results;
arrive at a first set of head health assessments based on the applying; and
report the first set of head health assessments.

2. The system of claim 1 wherein the hair analysis assembly comprises a mobile device and a hair analysis device that removably attaches to the mobile device.

3. The system of claim 1 wherein the hair analysis assembly is further configured to receive a product recommendation based on the first set of head health assessments and report the product recommendation.

4. The system of claim 1 wherein the first set of head images comprises a first set of hair images and the first set of head health features comprises a first set of hair health features.

5. The system of claim 4 wherein the first set of hair health features comprises thermal damage, mechanical damage and dryness.

6. The system of claim 5 wherein the head health rules comprise hair health rules wherein the hair health rules comprise comparing the quantities of each of the first set of hair health features in the hair images to a configurable threshold for each of the first set of hair health features.

7. The system of claim 1 wherein the first set of head images comprises a first set of scalp images and the first set of head health features comprises a first set of scalp health features.

8. The system of claim 7 wherein the first set of scalp health features comprises small flakes, medium and large flakes, flaky hair shaft, flaky scalp oil pooling, gooey hair shaft, and build-up.

9. The system of claim 8 wherein the head health rules comprise scalp health rules wherein the scalp health rules comprise comparing the quantities of each of the first set of scalp health features in the scalp images to a configurable threshold for each of the first set of scalp health features.

10. The system of claim 9 wherein the hair analysis device further comprises a cross polarized light, and wherein the obtaining is with the cross polarized light on for a first subset of the first set of scalp health features.

11. The system of claim 1 wherein the first set of head images comprises a first set of hair images and a first set of scalp images and the first set of head health features comprises a first set of hair health features and a first set of scalp health features.

12. The system of claim 11 wherein the first set of hair health features comprises thermal damage, mechanical damage and dryness and the first set of scalp health features comprises small flakes, medium and large flakes, flaky hair shaft, flaky scalp oil pooling, gooey hair shaft, and build-up.

13. The system of claim 12 wherein the head health rules comprise hair health rules, wherein the hair health rules comprise comparing the quantities of each of the first set of hair health features in the hair images to a configurable threshold for each of the first set of hair health features, and scalp health rules wherein the scalp health rules comprise comparing the quantities of each of the first set of scalp health features in the scalp images to a configurable threshold for each of the first set of scalp health features.

14. The system of claim 13 wherein the obtaining further comprises preparing the head analysis device for the head images to be obtained and head characteristics to be assessed by establishing a capabilities setup therefor.

15. A method for head health assessment of a user, the method comprising:
receiving, by a hair analysis assembly, an input to initiate the capture of a first set of head images of a user;
obtaining, by the hair analysis assembly, the first set of head images;
sending the first set of head images to a trained artificial intelligence system for analysis of the first set of head images for a first set of head health features;
receiving, by the hair analysis assembly from the trained artificial intelligence system, a first set of head health feature results comprising the locations and quantities of each of the first set of head health features in each image in the first set of user head images;
applying a first set of head health rules to the first set of head health feature results;
arriving at a first set of head health assessments based on the applying; and
report the first set of head health assessments.

16. The method of claim 15 wherein the first set of head images comprises a first set of hair images and the first set of head health features comprises a first set of hair health features.

17. The method of claim 16 wherein the first set of hair health features comprises thermal damage, mechanical damage and dryness.

18. The method of claim 17 wherein the head health rules comprise hair health rules wherein the hair health rules comprise comparing the quantities of each of the first set of hair health features in the hair images to a configurable threshold for each of the first set of hair health features.

19. The method of claim 15 wherein the first set of head images comprises a first set of scalp images and the first set of head health features comprises a first set of scalp health features.

20. The method of claim 19 wherein the first set of scalp health features comprises small flakes, medium and large flakes, flaky hair shaft, flaky scalp oil pooling, gooey hair shaft, and build-up.

21. The method of claim 20 wherein the head health rules comprise scalp health rules wherein the scalp health rules comprise comparing the quantities of each of the first set of scalp health features in the scalp images to a configurable threshold for each of the first set of scalp health features.

22. The method of claim 21 wherein the hair analysis device further comprises a cross polarized light, and wherein the obtaining is with the cross polarized light on for a first subset of the first set of scalp health features.

23. The method of claim 15 wherein the first set of head images comprises a first set of hair images and a first set of scalp images and the first set of head health features comprises a first set of hair health features and a first set of scalp health features.

24. The method of claim 23 wherein the first set of hair health features comprises thermal damage, mechanical damage and dryness and the first set of scalp health features comprises small flakes, medium and large flakes, flaky hair shaft, flaky scalp oil pooling, gooey hair shaft, and build-up.

25. The method of claim 24 wherein the head health rules comprise hair health rules, wherein the hair health rules comprise comparing the quantities of each of the first set of hair health features in the hair images to a configurable threshold for each of the first set of hair health features, and scalp health rules wherein the scalp health rules comprise comparing the quantities of each of the first set of scalp health features in the scalp images to a configurable threshold for each of the first set of scalp health features.

26. The method of claim 25 wherein the obtaining further comprises preparing the head analysis device for the head images to be obtained and head characteristics to be assessed by establishing a capabilities setup therefor.
